(19) United States
(12) Patent Application Publication
(10) Pub. No.: US 2010/0093843 A1
(43) Pub. Date: Apr. 15, 2010

(54) NOVEL NUTRACEUTICAL AND PHARMACEUTICAL COMPOSITIONS AND USE THEREOF FOR THE TREATMENT, CO-TREATMENT OR PREVENTION OF CARTILAGE DEGRADATION OR CARTILAGE DAMAGE IN JOINTS

(76) Inventors: Joseph Schwager, Basel (CH); Daniel Raederstorff, Flaxlanden (FR); Goede Schueler, Eimeldingen (DE)

Correspondence Address: NIXON & VANDERHYE, PC, 901 NORTH GLEBE ROAD, 11TH FLOOR, ARLINGTON, VA, 22203, US

(21) Appl. No.: 12/438,405

(22) PCT Filed: Sep. 12, 2007
(86) PCT No.: PCT/EP07/07925

§ 371 Date: Nov. 5, 2009

(30) Foreign Application Priority Data
Sep. 12, 2006 (EP) 06019070.9
Jan. 15, 2007 (EP) 07000686.1

(51) Int. Cl. A61K 31/352; C07D 311/86; A61P 19/00

(52) U.S. Cl. 514/455; 549/392

(57) ABSTRACT

The invention relates to novel nutraceutical and pharmaceutical compositions comprising at least a compound of the general formula (I), wherein R¹ and R² are independently from each other C₁₋₈-alk(en)yl and R³ and R⁴ are independently from each other hydroxy or C₁₋₈-alkyloxy, as well as to the use of these compositions for the treatment, co-treatment or prevention of joint disorders.

The invention relates to novel nutraceutical and pharmaceutical compositions comprising at least a compound of the general formula I as well as to the use of these compositions for the treatment, co-treatment or prevention of joint disorders or maintenance of joint health.

It has been found that the compositions are especially suitable for the treatment, co-treatment or prevention of cartilage degradation or cartilage damage in joints and as such for treatment of the cartilage degradation component of joint disorders, for example degenerative joints disorders such as osteoarthritis; or sport injuries.

Cartilage degradation is defined within the framework of the invention as a metabolic disorder of joint cartilage characterized by increased production of cartilage-degrading enzymes such as matrix metalloproteases.

Osteoarthritis is a chronic degenerative disease of the joint of non-inflammatory origin, which develops by wear and tear of the joints during aging and results in pain and diminished joint function. Symptoms of osteoarthritis include pain, stiffness and loss of mobility in one or more joints. Excessive joint loading increases the risk of osteoarthritis, hence osteoarthritis mostly affects the weight-bearing joints such as spine, knees and hips, but thumb and finger joints may also be affected. Joint disorders can also results from injury, i.e. microdamage or blunt trauma, fractures, damage to tendons, menisci or ligaments or can be the result of excessive mechanical stress or other biomechanical instability resulting from for example an injury or obesity.

Joint disorders due to cartilage degradation are leading causes of disability and dysfunction in the elderly; almost 80% of people over age 60 show some evidence of these disorders. Age, genetic factors, muscle disuse and weakness, trauma, obesity and anatomical abnormalities contribute to the development of the disorder.

Joint disorders are difficult to treat. Up till now, the symptoms were mainly treated with non-steroidal anti-inflammatory drugs. The drugs are given to control the pain and to restrain swelling, but do not prevent or treat damage to the cartilage. The patients experiencing severe cartilage damage frequently require surgery, including joint replacement surgery. Therefore, there is a great need for agents that treat or prevent cartilage loss and damage, which is now solved by the present invention.

In a first aspect, the invention relates to a use of at least a compound of the general formula I,

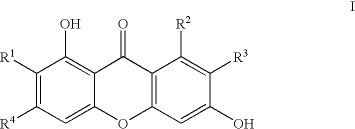

wherein R¹ and R² are independently from each other C₁₋₈-alk(en)yl and R³ and R⁴ are independently from each other hydroxy or C₁₋₈-alkyloxy, for the manufacture of a nutraceutical or pharmaceutical composition for the treatment, co-treatment or prevention of cartilage degradation or cartilage damage in joints, for cartilage regeneration or cartilage maintenance or for maintenance of joint health.

Preferred are compounds of the general formula I,

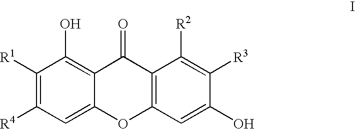

wherein R¹ and R² are independently from each other C₁₋₆-alk(en)yl and R³ and R⁴ are independently from each other hydroxy or C₁₋₆-alkyloxy, even more preferred are compounds of the general formula I, wherein R¹ and R² are independently from each other C₁₋₆-alk(en)yl and R³ and R⁴ are independently from each other hydroxy or methoxy.

As used in the context of the invention the term “alkyl” refers to, optionally substituted, straight chain or branched chain or cyclic hydrocarbon groups containing 1 to 8 carbon atoms, such as methyl, ethyl, propyl, isopropyl, butyl, sec. butyl, isobutyl, pentyl, neopentyl, hexyl, 2-ethyl-hexyl, cyclohexyl and octyl, preferably containing 1 to 6 carbon atoms. The optionally present substituents may be selected from halogen, hydroxy or alkoxy groups or by a mixture thereof. Preferably the alkyl group is unsubstituted.

“Alkenyl” refers to unsaturated straight chain or branched chain or cyclic hydrocarbon groups containing 1 to 8, preferably 1 to 6, carbon atoms such as allyl or isoprenyl.

“Alkoxy” refers to saturated straight chain or branched chain or cyclic ether groups (-O-alkyl) containing 1 to 8, preferably 1 to 6, carbon atoms such as methoxy, ethoxy, propoxy, butoxy, etc.

Most preferably, the compound of the formula I is one, wherein R¹ and R² are both isoprenyl and wherein R³ and R⁴ are both methoxy (this compound is called “α-mangostin”):

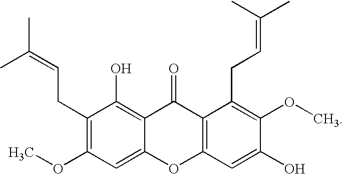

In preferred embodiments of the invention the compound of the formula I with the definitions of R¹ to R⁴ and the preferences as given above is the only compound of Garcinia mangostana L./mangosteen present in the compositions/medicaments.

The term “compound of the formula I” also encompasses any material or extract of a plant containing such a compound of the formula I. The amount of the compounds of the general formula I in a plant material or extract is not critical. Preferably the plant material or extract contains the compound of the general formula I in an amount of at least 1 weight-%, more preferably in an amount of at least 50 weight-%, even more preferably in an amount of at least 90 weight-%, based on the total weight of the plant material or extract. The terms “material of a plant” and “plant material” used in the context of the present invention mean any part of a plant.

α-Mangostin (1,3,6-trihydroxy-7-methoxy-2,8-bis(3-methyl-2-butenyl)-9H-xanthen-9-one) can be isolated from plants like Garcinia mangostana (see Journal of American Chemical Society 1958, 80, 1691 ff.; Phytochemistry 1996, 43(5), 1099-1102), but not limited to them.

Therefore, any material or extract of these plants or any other plant material or extract containing α-mangostin, preferably in an amount of at least 1 weight-%, more preferably in an amount of at least 50 weight-%, even more preferably in an amount of at least 90 weight-%, based on the total weight of the plant material or extract is also encompassed by this expression. “a-Mangostin” means both “natural” (isolated) and “synthetic” (manufactured) α-mangostin.

Beside the (pure) compound α-mangostin preferred are plant materials and plant extracts, especially those containing at least 10 weight-%, preferably at least 50 weight-%, more preferably at least 90 weight-%, of these compounds, based on the total weight of the plant material/extract.

The compositions according to the invention comprising at least a compound of the general formula I are especially attractive, since patients have a special interest in treatment considered as “natural” with mild effects and without major side-effects, because this composition comprising at least a compound of the general formula I according to the present invention can be used for disease prevention and as adjuvant treatment.

The compositions of the present invention are especially suitable for the treatment, co-treatment and prevention of cartilage degradation or cartilage damage in joints and as such for the treatment of the cartilage degradation component of joint disorders, for example degenerative joint disorders such as osteoarthritis; or sport injuries. The compositions of the present invention may have one or more of the following properties: it maintains and/or improves joint health, it prevents joint stiffness, it promotes joint mobility, it provides supple and/or flexible joints, it lubricates the joints, it relieves arthritis pain, it lessens joint problems, it provides joint care, it treats or prevents joint degradation, it provides joint integrity, it retards or prevents the progression of joint damage, it supports joint function, it promotes joint health and function, it naturally supports joint health and mobility for active individuals, it maintains the active flexibility of joints, it promotes joint flexibility and promotes joint mobility.

Preferably, the compound of the general formula I used in the compositions according to the invention is α-mangostin.

The compound(s) of the general formula I used in compositions according to the invention is/are preferably employed in a concentration so that at least 0.005 mg/kg bodyweight/day are administered to an animal including humans. Preferably, the daily consumption by an animal including human is in the range of 0.01 to 50 mg/kg bodyweight/day, most preferably in an amount of 0.1 to 15 mg/kg bodyweight/day based on the weight of the pure compound of the general formula I.

In the framework of the invention, with animals is meant all animals, including mammals, examples of which include humans. Preferred examples of mammals beside humans are non-ruminant or ruminant animals including cats, dogs, dromedaries, camels, elephants, and horses.

In another embodiment the invention relates to a nutraceutical composition comprising at least a compound of the general formula I and a nutraceutically acceptable carrier.

The term nutraceutical composition as used herein include food product, foodstuff, dietary supplement, nutritional supplement or a supplement composition for a food product or a foodstuff.

Preferably the nutraceutical composition is a dietary supplement, a beverage or a food or an animal feed such as pet food.

As used herein, the term food product refers to any food or feed suitable for consumption by humans or animals. The food product may be a prepared and packaged food (e.g., mayonnaise, salad dressing, bread, or cheese food) or an animal feed (e.g., extruded and pelleted animal feed, coarse mixed feed or pet food composition). As used herein, the term foodstuff refers to any substance fit for human or animal consumption. The term dietary supplement refers to a small amount of a compound for supplementation of a human or animal diet packaged in single or multiple dose units. Dietary supplements do not generally provide significant amounts of calories but may contain other micronutrients (e.g., vitamins or minerals). The term nutritional supplement refers to a composition comprising a dietary supplement in combination with a source of calories. In some embodiments, nutritional supplements are meal replacements or supplements (e.g., nutrient or energy bars or nutrient beverages or concentrates).

Food products or foodstuffs are for example beverages such as non-alcoholic and alcoholic drinks as well as liquid preparation to be added to drinking water and liquid food, non-alcoholic drinks are for instance soft drinks, sport drinks, fruit juices, such as for example orange juice, apple juice and grapefruit juice; lemonades, teas, near-water drinks and milk and other dairy drinks such as for example yoghurt drinks, and diet drinks. In another embodiment food products or foodstuffs refer to solid or semi-solid foods comprising the composition according to the invention. These forms can include, but are not limited to baked goods such as cakes and cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt and any other fat or oil containing foods, and food ingredients (e.g., wheat flour).

The term food products or foodstuffs also includes functional foods and prepared food products, the latter referring to any pre-packaged food approved for human consumption.

Animal feed including pet food compositions advantageously include food intended to supply necessary dietary requirements, as well as treats (e.g., dog biscuits) or other food supplements. The animal feed comprising the composition according to the invention may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

Dietary supplements of the present invention may be delivered in any suitable format. In preferred embodiments, dietary supplements are formulated for oral delivery. The ingredients of the dietary supplement of this invention are contained in acceptable excipients and/or carriers for oral consumption. The actual form of the carrier, and thus, the dietary supplement itself, is not critical. The carrier may be a liquid, gel, gelcap, capsule, powder, solid tablet (coated or non-coated), tea, or the like. The dietary supplement is preferably in the form of a tablet or capsule and most preferably in the form of a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical composition Sciences (Maack Publishing Co., Easton, Pa.).

In other embodiments, the dietary supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food. The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorings, inert ingredients, and the like.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolinate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising the composition according to the invention. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk, soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32. The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

It is understood by those of skill in the art that other ingredients can be added to those described herein, for example, fillers, emulsifiers, preservatives, etc. for the processing or manufacture of a nutritional supplement.

Additionally, flavors, coloring agents, spices, nuts and the like may be incorporated into the nutraceutical composition. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the nutraceutical compositions. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutraceutical composition can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The dosage and ratios of the composition according to the invention comprising at least a compound of the general formula I via a nutraceutical composition will, of course, vary depending upon known factors, such as the physiological characteristics of the particular combination and its mode and route of administration; the age, health and weight of the recipient; the nature and extent of the symptoms; the kind of concurrent treatment; the frequency of treatment; and the effect desired and can be determined by the expert in the field with normal trials, or with the usual considerations regarding the formulation of a nutraceutical composition.

Preferred are nutraceutical compositions comprising about 0.05 mg to 500 mg, preferably 2.0 mg to 300 mg of at least a compound of the general formula I per serving.

In another aspect, the invention relates to a pharmaceutical composition comprising at least a compound of the general formula I and a pharmaceutically acceptable carrier.

A person skilled in the art knows which carriers can be used as pharmaceutically acceptable carriers. Suitable pharmaceutical carriers are e.g. described in Remington's Pharmaceutical Sciences, supra, a standard reference text in this field. Examples of such pharmaceutically acceptable carriers are both inorganic and organic carrier materials, suitable for oral/parenteral/injectable administration and include water, gelatin, gum arabic, lactose, starch, magnesium stearate, talc, vegetable oils, and the like.

The pharmaceutical composition may further comprise conventional pharmaceutical composition additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

In a preferred embodiment the pharmaceutical composition is in the form of a powder, tablet, capsule, gel, liquid or solid embodiment.

The dosages and ratios of the individual components in a pharmaceutical composition can be determined by the expert in the field with normal preclinical and clinical trials, or with the usual considerations regarding the formulation of pharmaceutical composition.

In a preferred embodiment the compound(s) of the general formula I is/are administered via a pharmaceutical composition either in the form of a single dose or by multiple doses in an amounts of at least 0.005 mg/kg bodyweight/day, preferably in an amount of 0.01 to 50 mg/kg bodyweight/day, most preferably in an amount of 0.1 to 15 mg/kg bodyweight/day based on the weight of the pure derivatives. Thus, the pharmaceutical composition may for example comprise the compound(s) of the formula I in an amount from 1 mg to 500 mg per dosage unit, e.g. per capsule or tablet, of from 5 mg daily dose to 2000 mg per daily dose of a liquid formulation.

The nutraceutical and pharmaceutical compositions according to the present invention may be in any galenic form that is suitable for administering to the animal body including the human body, more in particular in any form that is conventional for oral administration, e.g. in solid form, for example as (additives/supplements for) food or feed, food or feed premixes, fortified food or feed, tablets, pills, granules, dragées, capsules, and effervescent formulations such as powders and tablets, or in liquid form, for instance in the form of solutions, emulsions or suspensions, for example as beverages, pastes and oily suspensions. The pastes may be filled into hard or soft shell capsules, whereby the capsules feature e.g. a matrix of (fish, swine, poultry, cow) gelatin, plant proteins or ligninsulfonate. Examples for other application forms are forms for transdermal, parenteral, topical or injectable administration. The nutraceutical and pharmaceutical compositions may be in the form of controlled (delayed) release formulations. Examples of pharmaceutical composition also include compositions suitable for topical application such as crèmes, gels, sprays, dry sticks, powders etc.

Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

Also, the invention relates to a method for treatment, co-treatment or prevention of cartilage degradation or cartilage damage in joints or for maintenance of joint health in animals including humans said method comprising the step of administering an effective amount of at least a compound of the general formula I,

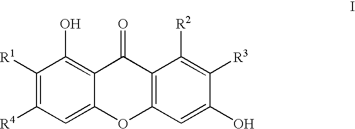

wherein R¹ and R² are independently from each other C₁₋₈-alk(en)yl and R³ and R⁴ are independently from each other hydroxy or C₁₋₈-alkyloxy, preferably wherein R¹ and R² are independently from each other C₁₋₆-alk(en)yl and R³ and R⁴ are independently from each other hydroxy or C₁₋₆-alkyloxy, even more preferably wherein R¹ and R² are independently from each other C₁₋₆-alk(en)yl and R³ and R⁴ are independently from each other hydroxy or methoxy, most preferably α-mangostin, to animals including humans, which are in need thereof.

The term “an effective amount of the composition according to the invention” refers to an amount necessary to obtain a physiological effect. The physiological effect may be achieved by one single dose or by repeated doses. The dosage administered may, of course, vary depending upon known factors, such as the physiological characteristics of the particular combination and its mode and route of administration; the age, health and weight of the recipient; the nature and extent of the symptoms; the kind of concurrent treatment; the frequency of treatment; and the effect desired and can be adjusted by a person skilled in the art.

### EXAMPLES

α-Mangostin is e.g. commercially available at Indofine Chemicals Company Inc, 121 Stryker Lane Hillsborough, N.J. 08844 or from APIN chemicals.

### Example 1

### Soft Gelatin Capsule

Soft gelatin capsules are prepared by conventional procedures providing a dose of at least a compound of the general formula I of 100 mg per capsule. A suitable daily dose is 1 to 5 capsules.

Other ingredients: glycerol, water, gelatine, vegetable oil.

### Example 2

### Hard Gelatin Capsule

Hard gelatin capsules are prepared by conventional procedures providing a dose of at least a compound of the general formula I in an amount providing a sum of 75 mg per capsule based on the pure derivatives. A suitable daily dose is 1 to 5 capsules.

Other ingredients:

Fillers: lactose or cellulose or cellulose derivatives q.s.

Lubricant: magnesium stearate if necessary (0.5%).

### Example 3

### Tablet

Tablets are prepared by conventional procedures providing as active ingredient 100 mg of at least a compound of the general formula I per tablet, and as excipients microcrystalline cellulose, silicone dioxide (SiO₂), magnesium stearate, crosscarmellose sodium ad 500 mg.

### Example 4

### Soft Drink

A soft drink containing a compound of the general formula I may be prepared from the following ingredients:

| | |
|---|---|
| ingredient | [g] |

A. juice concentrates and water soluble flavors

| | |
|---|---|
| 60.3°Brix, 5.15% acidity | 657.99 |
| 43.5° Brix, 32.7% acidity | 95.96 |
| Orange flavor, water soluble | 3.43 |
| Apricot flavor, water soluble | 6.71 |
| water | 26.46 |

B. color

| | |
|---|---|
| β-carotene 10% CWS | 0.89 |
| water | 67.65 |

C. Acid and antioxidant

| | |
|---|---|
| Ascorbic acid | 4.11 |
| Citric acid anhydrous | 0.69 |
| water | 43.18 |

D. stabilizers

| | |
|---|---|
| pectin | 0.20 |
| Sodium benzoate | 2.74 |
| water | 65.60 |

E. oil soluble flavors

| | |
|---|---|
| Orange flavor, oil soluble | 0.34 |
| Orange oil distilled | 0.34 |

F. active ingredient

| | |
|---|---|
| at least a compound | Amount providing a sum of 50 mg based |
| of the general | on the pure derivatives. |
| formula I | |

Fruit juice concentrates and water soluble flavors are mixed without incorporation of air. The color is dissolved in deionized water. Ascorbic acid and citric acid are dissolved in water. Sodium benzoate is dissolved in water. The pectin is added under stirring and dissolved while boiling. The solution is cooled down. Orange oil and oil soluble flavors are premixed. The active ingredient as mentioned under F is stirred into the fruit juice concentrate mixture of A.

In order to prepare the soft drinks all components A-F are mixed together before homogenizing using a Turrax and then a high-pressure homogenizer (p₁=200 bar, p₂=50 bar).

### Example 5

### Influence of α-Mangostin on the Expression of Genes Involved in the Build-Up and Break-Down of Cartilage

In articular cartilage, a delicate balance between anabolic (build-up) and catabolic (break down) events needs to be maintained in order to prevent hypertrophy and excessive degradation of extracellular matrix (ECM), respectively. The ECM is built up of collagen and proteoglycans that are the products of collagen genes, for example human collagen I and collagen II or aggrecan genes, which are activated and expressed during anabolic processes.

Catabolic events are controlled by the expression of genes, for example those genes that encode matrix metalloproteinases (MMPs) that eventually break down collagen or proteoglycans. Of the MMPs, MMP-1, MMP-3 and MMP-13 have a major role in breaking down the ECM in cartilage degradation.

α-Mangostin was obtained from APIN chemicals and contained according to the specification >98% of α-mangostin. In this experiment the effect of α-mangostin (12.5 and 6.25 μM) on the expression of human collagen I, II and aggrecan, as well as human MMP-3 and 13 was measured in vitro in a normal human articulate cells from knee (obtained from Cambrex). A control experiment without α-mangostin was done concomitantly to compare the expression of these genes to basal levels (the percentage of gene expression in control is set to 100%).

The cells were treated with IL-1β, which is one of the natural mediators that induce cartilage breakdown and is detected in substantial amounts in osteoarthritic cartilage tissues and cells derived thereof. Treatment of cells with IL-1β triggers the expression of genes that are involved in catabolic events, such as MMPs. Also, treatment of the cells with IL-1β reduces the collagen expression levels (compared to untreated cells).

Normal human articulate cells (purchased from the Cambrex) were grown in cell culture medium according to the instructions obtained from the manufacturer. For experimental treatments, cells were seeded into 6-well plates and cultured for two days until they nearly reached confluence. Cells were then activated with IL1-β (10 ng/ml) with the indicated amounts of α-mangostin and incubated for 4 hours. Total cellular RNA was extracted from cultured cells and reverse-transcribed as detailed by Richard et al. Mol. Nutr. Food Res. 49, 431-442, 2005. Expression levels of distinct genes were determined by quantitative real-time PCR as also described in Richard et al. Mol. Nutr. Food Res. 49, 431-442, 2005. Results of this experiment are shown in Table 1 below:

TABLE 1
Effect of α-mangostin on gene expression in human chondrocyte cells
(SW1353).

| | 0 μM α- | 12.5 μM α- | 6.25 μM α- |
|---|---|---|---|
| Gene | mangostin | mangostin | mangostin |
| Human MMP-3 | 100% | 73% | 78% |
| Human MMP-13 | 100% | 77% | 73% |
| Human collagen I | 100% | 105% | 126% |
| Human collagen | 100% | 247% | 127% |
| II | | | |
| Aggrecan | 100% | 157% | 172% |

α-Mangostin drastically and dose-dependently reduced the expression of genes contributing to the degradation of cartilage (MMP-3 and MMP-13), whereas it stimulated the expression of some genes associated with the build-up of cartilage (human collagen I, human collagen II, aggrecan). These results suggest that α-mangostin not only prevents degradation of cartilage, but also helps to regenerate cartilage tissue.

1. Use of at least a compound of the general formula I, 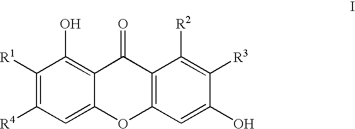
wherein R¹ and R² are independently from each other C₁₋₈-alk(en)yl and R³ and R⁴
are independently from each other hydroxy or C₁₋₈-alkyloxy, for the manufacture of a nutraceutical or pharmaceutical composition for the treatment, co-treatment or prevention of cartilage degradation or cartilage damage in joints, for cartilage regeneration or cartilage maintenance, or for maintenance of joint health.

2. The use as in claim 1, wherein R¹ and R² are independently from each other C₁₋₆-alk(en)yl and R³ and R⁴ are independently from each other hydroxy or C₁₋₆-alkyloxy.

3. The use as in claim 1, wherein R¹ and R² are independently from each other C₁₋₆-alk(en)yl and R³ and R⁴ are independently from each other hydroxy or methoxy.

4. The use as in claim 1, wherein the compound of the general formula I is α-mangostin.

5. A nutraceutical composition comprising at least a compound of the general formula I as defined in claim 1 and a nutraceutically acceptable carrier.

6. The nutraceutical composition as in claim 5 which is a dietary supplement, a beverage or a food or an animal feed.

7. The nutraceutical composition as in claim 5 in which the amount of the at least a compound of the general formula I as defined in claims 1 to 4 is in the range of 0.05 mg to 500 mg, preferably in the range of 2.0 mg to 300 mg per serving.

8. A pharmaceutical composition comprising at least a compound of the general formula I as defined in claim 1 and a pharmaceutically acceptable carrier.

9. A method for treatment, co-treatment or prevention of cartilage degradation or cartilage damage in joints, for cartilage regeneration or cartilage maintenance, or for maintenance of joint health, in animals including humans, said method comprising the step of administering an effective amount of at least a compound of the general formula I as defined in claim 1 to animals including humans, which are in need thereof.

10. The method as in claim 9 wherein the animals including humans are selected from the group consisting of humans, dogs, cats, horses, and camels.
